(19) United States
(12) Patent Application Publication
(10) Pub. No.: US 2011/0237910 A1
(43) Pub. Date: Sep. 29, 2011

(54) STABILIZED MULTI-WAVELENGTH LASER SYSTEM FOR NON-INVASIVE SPECTROPHOTOMETRIC MONITORING

(75) Inventors: John K. Gamelin, Avon, CT (US); Paul B. Benni, Acton, MA (US)

(73) Assignee: CAS MEDICAL SYSTEMS, INC., Branford, CT (US)

(21) Appl. No.: 13/070,172

(22) Filed: Mar. 23, 2011

Related U.S. Application Data
(60) Provisional application No. 61/316,633, filed on Mar. 23, 2010.

(51) Int. Cl. A61B 5/1455

(52) U.S. Cl. 600/323

(57) ABSTRACT

A spectroscopic method and system that monitors oxygenation levels in biological tissue is provided. The system includes a sensor portion, a monitor portion, and at least one optical fiber light stabilizer. The sensor portion includes at least one sensor assembly, which sensor assembly has at least one light signal outlet, and at least one light detector adapted to sense light and produce detected signals. The monitor portion has a processor in communication with the light detector in the sensor assembly, and a light source adapted to produce laser light signals at a plurality of different wavelengths. The optical fiber light stabilizer is adapted to stabilize the laser light signals. The processor is adapted to process the detected signals to determine oxygenation levels within the biological tissue.

Applicant hereby claims priority benefits under 35 U.S.C. §119(e) of U.S. Provisional Patent Application No. 61/316,633 filed Mar. 23, 2010, the disclosure of which is herein incorporated by reference.

### BACKGROUND OF THE INVENTION

1. Technical Field

This invention relates in general to apparatus and methods for non-invasively examining biological tissue utilizing near-infrared spectroscopy techniques, and in particular to a relatively stabilized laser diode light source for use with such apparatus and methods.

2. Background Information

Near-infrared spectroscopy (NIRS) is an optical spectrophotometric method that can be used to continuously monitor biological tissue characteristics such as the oxygenation level within the tissue. The NIRS method is based on the principle that light in the red/near-infrared range (660-1000 nm) can pass easily through skin, bone and other tissues where it encounters hemoglobin located mainly within micro-circulation passages; e.g., capillaries, arterioles, and venuoles. Hemoglobin exposed to light in the near-infrared range has specific absorption spectra that vary depending on its oxygenation state; i.e., oxyhemoglobin (HbO₂) and deoxyhemoglobin (Hb) each act as a distinct chromophore. By using light sources that transmit near-infrared light at specific different wavelengths, and by measuring changes in transmitted or reflected light attenuation, concentration changes of the oxyhemoglobin and deoxyhemoglobin can be monitored as well as total or absolute values of tissue oxygenation levels can be determined or calculated. The ability to continually monitor or determine cerebral oxygenation levels, for example, is particularly valuable for those patients subject to a condition in which oxygenation levels in the brain may be compromised, leading to brain damage or death.

A NIRS system typically includes a sensor portion having a light source and one or more light detectors for detecting reflected and/or transmitted light. The light signal is created and sensed in cooperation with the overall NIRS system that includes a monitor portion having a computer or processor that runs an algorithm for processing signals and the data contained therein to, for example, calculate or determine the hemoglobin oxygenation concentration or saturation levels. Typically the monitor portion is separate from the sensor portion. Light sources such as light emitting diodes (LEDs) or laser diodes that produce light emissions in the wavelength range of 660-1000 nm are typically used. Each light source produces an infrared light signal at a particular wavelength at which a known absorption response is produced depending on the amount of oxygen concentration in the hemoglobin. Several different specific wavelengths are typically employed, for example, at 690 nm, 780 nm, 805 nm, and 850 nm. Thus, a corresponding number of light sources are employed in the sensor portion, with these light sources usually being located together. One or more photodiodes or other types of light detectors detect light reflected from or passed through the tissue being examined, and oftentimes the photodiodes are located at specific, predetermined different distances from the light source location. The NIRS system processor cooperates with the light source and detector to create, detect and analyze the signals, for example, in terms of their intensity and wave properties. U.S. Pat. Nos. 6,456,862 and 7,072,701, both of which are hereby incorporated by reference in their entirety, each disclose a NIRS system (e.g., a cerebral oximeter) and a methodology for analyzing the signals within the NIRS system to produce an indication of tissue oxygenation levels to a system user, typically a clinician.

However, a spectrophotometric system such as a cerebral oximeter that utilizes a laser system containing one or more laser diodes may demonstrate instability in operation for various reasons. For example, the output of the laser system may become unstable over time in terms of its wavelength and power output due to various factors, including environmental (e.g., temperature). Also, oftentimes the individual laser diode(s) is located apart from the sensor portion of the overall system and, as such, the laser diode output may be coupled directly by an optical fiber to the sensor portion. Therefore, problems may exist, for example, in the connection or coupling of the laser diode output to the optical fiber, for example, due to stripped cladding of the optical fiber, improper centering of the optical fiber in the connector, or use of an improper connector. Also, instabilities in the optical fiber itself may exist, for example, due to bending, temperature, and mode variance. In general, it is known that when the sensor portion of the spectrophotometric system is directly connected to the laser diode light source by an optical fiber of a few meters in length, an unstable light output can occur. Any sufficient degree of instability in the overall laser system output can cause corresponding errors in the overall spectrophotometric system, particularly those that utilize differential wavelength algorithms.

In addition, some oximetry monitors are multi-channel devices with a plurality of sensor portions, with each channel being associated with a different particular sensor portion. These devices often employ parallel sets of optical, electronic, and software subsystems, each dedicated to a different channel/sensor portion. The “parallel” approach, although straightforward to implement, can have several disadvantages. For example, replication of hardware in each parallel channel increases the cost, the size, and the complexity of the device, and the opportunity for a component to fail. In addition, each channel will have its own distinct spectrophotometric characteristics such as central wavelength, spectral bandwidth, and modal profile. These characteristics can vary from channel to channel, and can also vary independently as a function of time and environmental conditions. These variances in spectrophotometric characteristics will very likely result in measurement discrepancies between channels. The variances per channel within the monitor portion may also be additive to variances in the sensor portion for each channel.

What is needed, therefore, is a laser system light source that contains multiple light sources that can provide different discrete wavelengths for use in a spectrophotometric system such as a cerebral oximeter, where the laser system provides a relatively stable and consistent light radiation output in terms of output parameters such as, for example, power, intensity and radiation pattern, and a system that provides uniformity between channels.

### SUMMARY OF THE INVENTION

According to the present invention, a spectroscopy system that may be used for spectrophotometric monitoring of tissue includes a monitor portion and a sensor portion. The spectroscopy system is described hereinafter as a cerebral oximeter operable to monitor brain tissue. The spectroscopy system is not limited to a cerebral oximeter embodiment, however, and may be utilized in other spectroscopic applications. The sensor portion generally includes one or more channels, each having a light source and one or more light detectors. The sensor portion may attach to a human to sense light signals from the light source that have traversed biological tissue, the light signals ultimately being used by the system to determine biological tissue blood hemoglobin oxygenation levels. The monitor portion generally includes a processor for determining or calculating tissue oxygenation levels from the sensed light signals, together with a visual display to indicate the determined oxygenation levels in various forms. The light source may comprise a plurality of laser diodes, LEDs, or the like, each providing infrared light at a particular wavelength. A laser beam combiner may couple the plurality of laser diode output light signals into one optical fiber. To stabilize the output of each of the laser diodes in the laser beam combiner, an optical fiber light stabilizer may be coupled to the combined laser diode output. The light stabilizer may include several meters of multimode optical fiber wrapped around a circular spool. The optical fiber coupled to a laser diode is typically “underfilled” when the laser light enters the optical fiber (i.e., usually only the lower-order propagation modes or paths are utilized in the optical fiber) since the laser diode radiation output has a lower numeral aperture (NA) compared to the optical fiber. The optical fiber light stabilizer redistributes the propagation modes so that the higher-order modes are filled until an equilibrium mode distribution is established. The propagation modes nearest to the axis of the fiber core are referred to as the lower-order modes, while the paths with the relatively greatest deviation (i.e., highest angles from the core axis) are referred to as the higher-order modes. The resultant laser system light output typically demonstrates a relatively high degree of stability when modal equilibrium is achieved. A light sensor (e.g., a photodiode) may also provide feedback with respect to the laser diode output, which allows for compensation of any laser diode light output instability independently of optical fiber related instabilities.

With an underfilled optical fiber, the light may “jump” between lower and higher modes due to temporary fiber bending or temperature changes, which causes instability in the laser system output. The optical fiber light stabilizer corrects this problem by redistributing the light into an equilibrium mode distribution. Such an equilibrium mode distribution may also be achieved with a relatively large amount (e.g., 1000-2000 meters) of uncoiled optical fiber. For example, a laser diode connected to a multimode optical fiber cable a few meters in length with a numeral aperture (NA) of 0.22 (conic light output of 12.7 degrees) will fill only the lower modes, resulting in an output NA of 0.18 (10.4 degrees) or less, depending on the laser light launch NA. By attaching the laser diode to the optical fiber light stabilizer comprising the same optical fiber but at a longer length (e.g., 20 meters) wrapped around a spool with a radius that is at least approximately equal to (e.g., or slightly larger than) the minimum long term bend radius of the optical fiber, the light output will reach an equilibrium mode distribution with an output NA of approximately 0.22 (12.7 degrees), resulting in a relatively more stable output.

According to an aspect of the present invention, a spectroscopic system that monitors oxygenation levels in biological tissue is provided. The system includes a sensor portion and a monitor portion. The sensor portion includes a plurality of sensor assemblies. Each sensor assembly has at least one light signal outlet, and at least one light detector adapted to sense light and produce detected signals. The monitor portion includes a processor, a light source adapted to produce light signals at a plurality of different wavelengths, and an optical switch with two or more output ports and an input port. Each switch output port is adapted to be connected to the light signal outlet of a particular one of the sensor assemblies. The switch input port is in communication with the light source. In some embodiments, the monitor portion includes a multiplexer having a plurality of input ports and an output port. Each multiplexer input port is adapted to be connected to a particular one of the light detectors in each sensor assembly. The multiplexer output port is in communication with the processor. The optical switch is adapted to selectively route light signals from the light source to each switch output port. The processor is adapted to process the detected signals to determine oxygenation levels within the biological tissue.

According to another aspect of the present invention, a method for spectrophotometrically determining an oxygenation level in biological tissue of a human subject is provided. The method includes the steps of: a) providing a plurality of laser output signals each at a predetermined wavelength of light; b) combining the plurality of laser output signals into a combined laser output signal; c) providing the combined laser output signal to an optical fiber, where the combined laser output signal propagates through the optical fiber; d) stabilizing the combined laser output signal within the optical fiber by redistributing modes of the combined laser output signal until an equilibrium mode distribution is established in the combined laser output signal propagating within the optical fiber; e) passing the stabilized laser output signal through an optical switch having a plurality of output ports; f) directing the laser output signal from each switch output port to a channel dedicated to that output port; g) selectively emitting the laser output signal from each channel into particular regions of biological tissue of the human subject; h) sensing for the laser output signal after it has passed through the biological tissue of the human subject, and producing detected signals corresponding to sensed laser output signals; i) multiplexing the detected signals into a processor; and j) determining the oxygenation level in the region of biological tissue of the human subject associated with each channel using the detected signals within the processor.

According to another aspect of the present invention, a method for spectrophotometrically determining an oxygenation level in biological tissue of a human subject is provided. The method includes the steps of: a) selectively providing a plurality of laser output signals each at a predetermined wavelength of light; b) providing the laser output signals to an optical fiber, where the laser output signals propagate through the optical fiber; c) stabilizing the laser output signals within the optical fiber by redistributing modes of the laser output signals until an equilibrium mode distribution is established in the laser output signals propagating within the optical fiber; d) emitting the laser output signals into the biological tissue of the human subject; e) sensing for the laser output signals after they have passed through the biological tissue of the human subject, and producing detected signals corresponding to sensed laser output signals; and f) determining the oxygenation level in the region of biological tissue of the human subject using the detected signals and a processor adapted to process the detected signals to determine the oxygenation level.

According to an aspect of the present invention, a spectroscopic system that monitors oxygenation levels in biological tissue is provided. The system includes a sensor portion, a monitor portion, and at least one optical fiber light stabilizer. The sensor portion includes at least one sensor assembly, which sensor assembly has at least one light signal outlet, and at least one light detector adapted to sense light and produce detected signals. The monitor portion has a processor in communication with the light detector in the sensor assembly, and a light source adapted to produce laser light signals at a plurality of different wavelengths. The optical fiber light stabilizer is adapted to stabilize the laser light signals. The processor is adapted to process the detected signals to determine oxygenation levels within the biological tissue.

These and other features and advantages of the present invention will become apparent in light of the drawings and detailed description of the present invention provided below.

### BRIEF DESCRIPTION OF THE DRAWINGS

FIG. 1 is a simplified diagrammatic representation of a NIRS system sensor portion placed on the head of a human subject and coupled to a NIRS system monitor portion.

FIG. 2 is a diagrammatic cross-section of the NIRS system sensor portion of FIG. 1.

FIG. 3 is a diagrammatic representation of an embodiment of a portion of the NIRS system of FIG. 1, including a combined multiple laser diode light source and associated components.

FIG. 4 is a detailed diagrammatic representation of an optical fiber light stabilizer assembly within the NIRS system of FIG. 3.

FIG. 5 is a diagrammatic representation of an embodiment of a portion of the NIRS system of FIG. 1, including a combined multiple laser diode light source and associated components.

FIG. 6 is a diagrammatic representation of a multi-channel embodiment of a portion of the NIRS system of FIG. 1, including a monitor portion having a single light source and detection configuration, an optical switch, and a multiplexer.

FIG. 7 is a diagrammatic representation of a multi-channel embodiment of a portion of the NIRS system similar to that shown in FIG. 6, further including a laser output intensity monitor disposed within the monitor portion.

FIG. 8 is a diagrammatic representation of a multi-channel embodiment of a portion of the NIRS system similar to that shown in FIG. 6, further including a laser output intensity monitor associated with each sensor assembly within the sensor portion.

FIG. 9 is a diagrammatic representation of a multi-channel embodiment of a portion of the NIRS system similar to that shown in FIG. 6, further including a laser output intensity model disposed within the monitor portion associated with the optical switch.

### DETAILED DESCRIPTION OF THE INVENTION

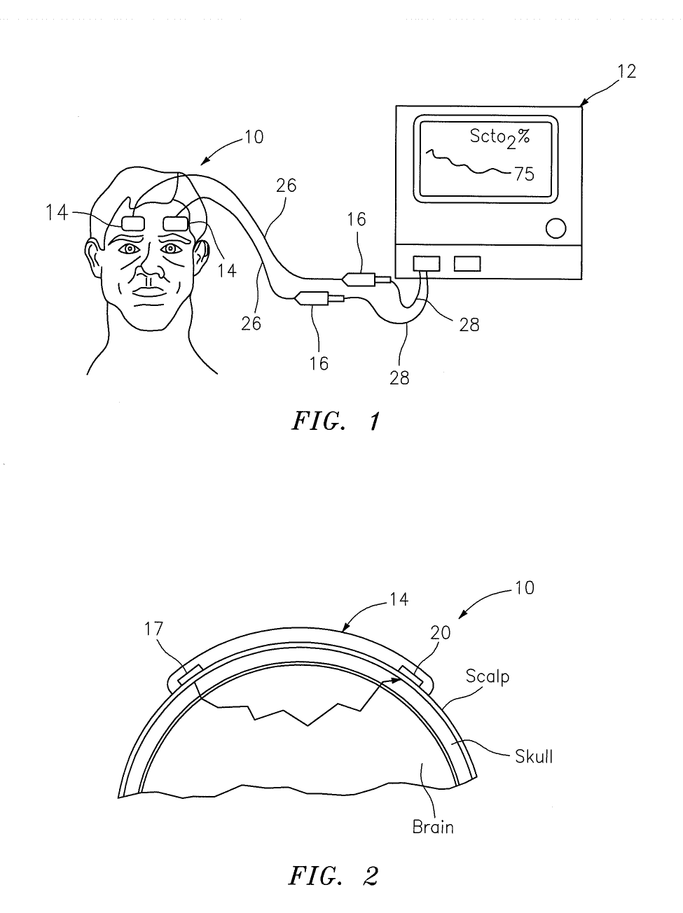

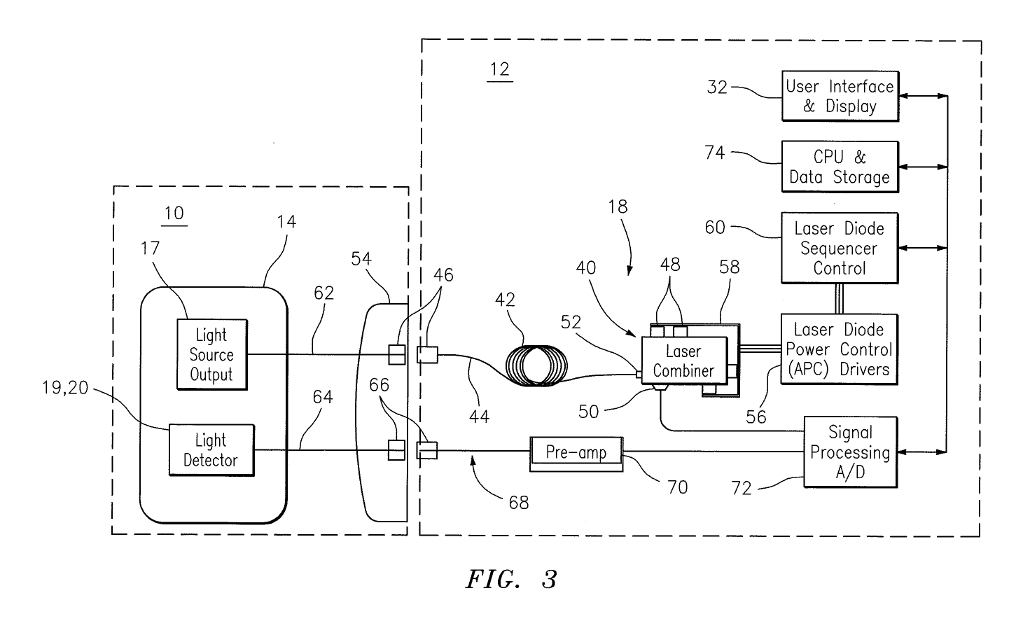

Referring to FIGS. 1 and 2, a NIRS spectrophotometric system for use with the present invention may be similar to that described and illustrated in the aforementioned U.S. Pat. Nos. 6,456,862 and 7,072,701. However, it should be understood that the present invention is not limited to use with the spectrophotometric systems of these patents, or with any specific spectrophotometric system. Instead, the present invention may be utilized with various types of spectroscopy apparatus or methods that include one or more laser diodes, LEDs, or other light emitting electro-optical components as light sources. The spectrophotometric system of FIGS. 1 and 2 generally includes a sensor portion 10 and a monitor portion 12. The sensor portion 10 may include one or more sensor assemblies 14 and one or more connector housings 16. Each sensor assembly 14, which may be a flexible structure that can be attached directly to a location (e.g., the head) on a human subject, may include a light source 18 and one or more light detectors 20. FIG. 2 diagrammatically illustrates a sensor assembly 14 having a single detector 20. FIG. 3 diagrammatically illustrates a sensor assembly having two detectors 19, 20; e.g., a near detector 19 and a far detector 20. An example of an acceptable sensor assembly 14 having more than one detector can be found in U.S. Publication No. 2009/0182209, which application is commonly assigned with the present application, and which is hereby incorporated by reference in its entirety. A disposable adhesive envelope or pad may be used for mounting the sensor assembly 14 easily and securely to the skin of the human subject under test. The light source 18 may comprise a plurality of laser diodes that in general emit a light signal at a narrow spectral bandwidth at known but different wavelengths (e.g., 690 nm, 780 nm, 805 nm, and 850 nm). The laser diodes are located within the monitor portion 12 (laser diodes 48 as will be described in more detail hereinafter), and have their light output transported to the sensor assembly 14 within the sensor portion 10 by way of an optical fiber cable. The light signals from the laser diodes exit the sensor assembly 14 at a light source outlet 17. A first connector cable 26 (see FIG. 1) connects each one of the sensor assemblies 14 to the connector housing 16, and a second connector cable 28 connects the connector housing 16 to the monitor portion 12. The light detector 20 may comprise photodiodes. Depending on the location of the laser diodes (i.e., in the sensor portion 10 or in the monitor portion 12), the connector cables 26, 28 may comprise only electrical cables or a combination of electrical and optical fiber cables. The monitor portion 12 includes an internal computer processor for processing light intensity signals from the light detector(s) 20 in accordance with various algorithms, for example those described in the aforementioned U.S. Pat. Nos. 6,456,862 and 7,072,701. The spectrophotometric system monitor portion 12 may include a display screen for visually displaying various types of information (e.g., the determined oxygen concentration or saturation levels) to the system user (e.g., a clinician).

Referring to FIG. 3, the spectrophotometric system monitor portion 12 includes various components, among them being a multiple laser beam combiner 40, an optical fiber light stabilizer 42, a predetermined length of multimode optical fiber 44, and an optical fiber connector coupler 46 (e.g., an ST-type connector coupler). The embodiment shown in FIG. 3 depicts a spectrophotometric system having a sensor portion 10 configured for a single sensor assembly 14. In those embodiments where the sensor portion 10 of the system is adapted for multiple sensor assemblies, the configuration of the aforesaid components for the single sensor assembly would be repeated for each of the additional sensor assemblies 14.

The multiple laser beam combiner 40 includes a plurality of laser diodes 48, a laser output monitor photodiode 50, and a fiber optic connector 52 (e.g., an SMA-type connector). An example of an acceptable combiner 40 is the multiple laser beam combiner provided by Princetel, Inc. of Lawrenceville, N.J., U.S.A. The Princetel laser beam combiner 40 typically has three or four laser diodes 48, and all of the laser diode light output signals are combined into a single laser beam or output light signal using beamsplitters and polarizing filters within the combiner 40. A lens inside the combiner 40 focuses the laser light output into the optical fiber 44 via the SMA connector 52. Alternative versions of the fiber optic connector 52 could be used, such as an APC connector, which reduces back reflection of light entering back into the laser combiner 40, potentially causing interference to laser diode power control and monitoring. An APC connector has an angled (e.g. about 8 degrees) polished fiber optic face, which redirects back reflected light in a different direction or axis from the output light signal that is entering into the APC connector by internal reflection. For example, an SMA connector could be polished at an angle of 8 degrees to function as an APC connector. The NIRS sensor assembly 14 optically interfaces to the spectrophotometric system monitor portion 12 via the optical fiber connector coupler 46, which may be part of a detachable connector 54 that connects the monitor portion 12 with the sensor portion 10. The detachable connector 54 may be part of the connector housing 16 of FIG. 1, or may be separate therefrom. During operation, the laser diodes 48 may be pulsed one at a time (time multiplexed) or pulsed at different frequencies (frequency multiplexed) and their light outputs are optically coupled to the multimode optical fiber 44 via the fiber optic connector 52. Laser light optically coupled to the multimode optical fiber 44 characteristically has a lower NA compared to that of the multimode optical fiber 44, and therefore the laser light usually underfills the modal structure of the optical fiber 44. The laser light propagates through the optical fiber light stabilizer 42, which effectively establishes equilibrium modal distribution in the multimode optical fiber 44. Equilibrium modal distribution is typically defined as the condition in a multimode optical fiber 44 where after light propagation has taken place for a certain distance down the fiber 44, known as the “equilibrium length,” the relative power distribution among modes becomes statistically constant and remains so for the duration of further propagation down the optical fiber. After the equilibrium length has been traversed, the NA of the output of the optical fiber 44 is independent of the NA of the light source (e.g., the laser diode) that sends light down the optical fiber. The laser light then propagates to the NIRS sensor assembly 14 through the remaining portion of the multimode optical fiber 44 and through the optical fiber connector coupler 46 to the sensor portion 10.

The laser diodes 48 are electrically actuated by laser diode power control drivers 56 via an electrical cable harness 58. A laser diode sequencer control 60 connects to the laser diode drivers 56 to provide laser diode pulse timing and control. The laser light from the multiple laser beam combiner 40 propagates through the optical fiber light stabilizer 44 and through the optical fiber connector coupler 46 to the NIRS sensor assembly 14. In the sensor assembly 14, the laser diode light propagates through a single core multimode optical fiber cable 62. The laser diode light is emitted out of the sensor assembly 14 at the light source outlet 17 and into the human subject (FIG. 1). The light detector 20 of the NIRS sensor assembly 14 receives the light after it has passed through the human subject being monitored via transmission and/or reflectance. The light detector 20 is electrically connected to a shielded cable 64 which interfaces with the NIRS monitor portion 12 via a shielded cable coupler 66. In some embodiments, the electrical signals received from the light detector 20 on a line 68 are electrically processed and amplified by a pre-amplifier 70 and by a signal processor 72, which may include an analog-to-digital converter. The signal processor 72 and CPU or monitor processor 74 convert the received signals into physiological parameters by various spectrophotometric methods (e.g., those of U.S. Pat. Nos. 6,456,862 and 7,072,701), and the resultant physiological parameters (e.g., tissue oxygenation concentration or saturation levels) may be visually displayed on the user display 32. Also, light sampled by the laser output monitor photodiode 50 could be used as the input intensity (Io) signal utilized in spectrophotometric type algorithms such that described in U.S. Pat. No. 6,456,862.

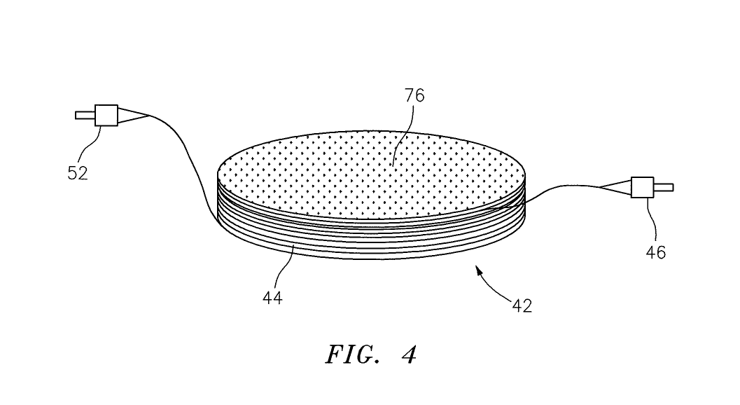

Referring to FIG. 4, there illustrated in more detail is the optical fiber light stabilizer 44, which may comprise the multimode optical fiber 44 wrapped around a circular spool 76. This may be carried out in a manner similar to the mandrel wrapping technique. A cylindrical rod wrap includes a specified number of turns of optical fiber on a mandrel or spool of a predetermined size, depending on the fiber characteristics and the desired modal distribution. Mandrel wrapping has application in optical transmission performance tests, to simulate or establish equilibrium mode distribution in a launch fiber (i.e., an optical fiber used to inject a test signal in another optical fiber under test). If the launch optical fiber is fully filled ahead of the mandrel wrap, the higher-order modes will be stripped off, leaving only the lower-order modes. If the launch optical fiber is underfilled, for example, as a consequence of being energized by a laser diode, there will be a redistribution to higher-order modes until modal equilibrium is reached. The spool 76 may have a radius that is at least approximately equal to the long term bend radius of the multimode optical fiber 44. One end of the multimode optical fiber 44 may be terminated by the fiber optic connector 52, and the other end of the multimode optical fiber 44 may be terminated by the optical fiber connector coupler 46. Other alterations besides that described above in a multimode optical fiber could be carried out to achieve equilibrium mode distribution. For example, a short segment of optical fiber placed in a rigid fixture that applies pressure on the fiber in different locations to cause microbends may be used, where such microbends induce redistribution of the modes to fill the higher-order modes until an equilibrium mode distribution is established. A mode scrambler could also be used to create an equilibrium mode distribution. An example of an acceptable type of mode scrambler is one where a relatively short piece of step index fiber is spliced into the optic fibers; e.g., between two sections of graded index fiber. Also, a combination of lenses may be used to achieve similar results.

The optical fiber light stabilizer 42, which is relatively rugged mechanically, provides for a relatively stable and consistent laser diode light output in terms of parameters such as power, intensity, and radiation pattern, which helps to ensure accuracy of NIRS system monitored parameters. For example, the relatively high degree of output light stability allows for accurate differential wavelength tissue oxygenation signal processing, such as that described in the aforementioned U.S. Pat. No. 6,456,862. Due to the increased output light stability, another advantage is that the discrete laser diode light output wavelengths may be spaced relatively closer together, which provides for relatively accurate tissue oxygenation spectrophotometric measurement, despite the closer wavelength dependent light absorption coefficient values. Closer spaced wavelengths also allow for relative reduction of wavelength dependent light pathlength differences, which may cause errors in tissue oxygenation spectrophotometric measurements. Another advantage is that different discrete wavelengths of light from the laser diodes 48 may be combined and interfaced to a single core multimode output optical fiber 44. Further, the different discrete wavelengths of light may pass through the optical fiber 44 in a homogeneous manner, such that the output light intensity from the single core multimode optical fiber 44 for all wavelengths is proportional to the input light intensity for all wavelengths, even if the input radiation profile or input NA are different for each wavelength. Still further, a homogeneous and relatively stable light output radiation profile or output NA may be achieved, even if the input radiation profile or input NA are lower and individually different for each wavelength. This is done by providing for relatively constant and high optical fiber modal filling by spreading the lower input modes to also fill higher modes until the optical fiber modes are filled; that is, a relatively large number of all of the modes or possible light guide pathways in the optical fiber 44 are utilized. Another advantage is that relatively homogeneous and stable light output intensity may be provided during rapid, transient, or gradual temperature changes, or during rapid, transient, or gradual optical fiber mechanical stress, such as fiber bending or vibration. A further advantage is that different optical sensors, each of a particular configuration used for biological tissue oxygenation measurement, may be interchangeably utilized without having to be individually calibrated.

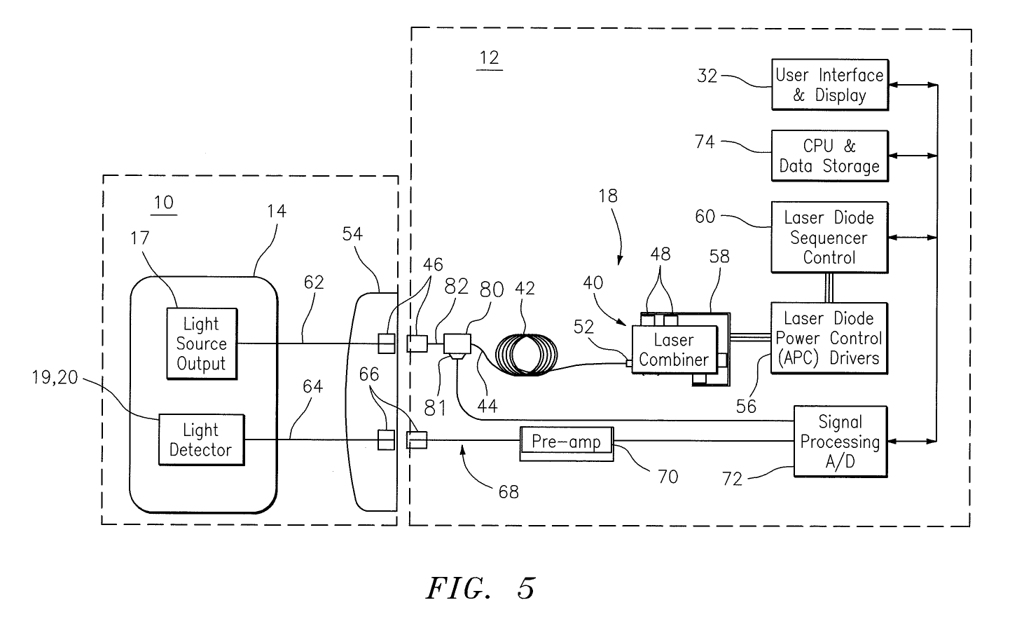

In an alternative embodiment, as shown in FIG. 5, an inline laser output intensity monitor 80 is placed after the optical fiber light stabilizer 42 to sample light using monitor photodiode 81, which functions in a manner similar to the laser output monitor photodiode 50 shown in FIG. 3. The inline laser output intensity monitor 80 contains a beam splitter that diverts a small percentage (e.g., ˜4%) of light to monitor photodiode 81. The diverted light can be used as an input intensity (Io) signal within spectrophotometric type algorithms such as that described in U.S. Pat. No. 6,456,862. In addition, the inline laser output intensity monitor can be used to increase the output NA of the light emitting from the sensor by a combination of lenses or other optical means. In such cases, the optical fibers 82 and 62 of the sensor would have a higher NA than the optical fiber used for the optical fiber light stabilizer 42. A higher NA output of the sensor advantageously improves safety margin when using laser light for spectroscopic examination of biological tissue by decreasing intensity over a given surface area.

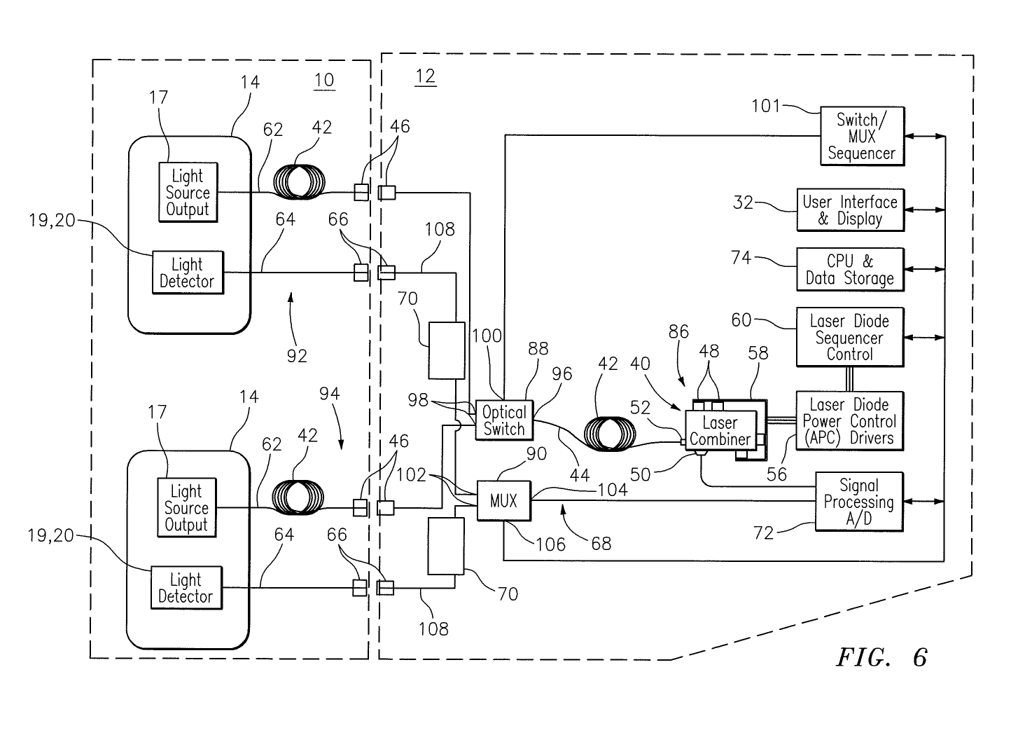

Referring to FIGS. 6-9, in alternative embodiments the spectrophotometric system is a multi-channel system wherein the monitor portion 12 includes a single light source 86 and detection configuration like that described above and shown in FIGS. 3 and 5 (e.g., one configuration that includes a multiple laser beam combiner 40, an optical fiber light stabilizer 42, a predetermined length of multimode optical fiber 44, a signal processor 72, etc.), and in addition also includes an optical switch 88 and a multiplexer 90 (or “MUX”). In these embodiments, rather than have a separate light source and detection configuration for each channel, the invention utilizes one light source and detection configuration for all of the channels 92, 94. As a result, each channel 92, 94 receives light signals having the same characteristics from the same highly stabilized light source 86. The uniformity of the sensor interrogation signals improves the overall accuracy and the site-comparative accuracy by enforcing identical algorithm spectral bias points and eliminating means of variation that may arise from source mode hopping, different spectral profiles, or spatial mode characteristics of the sensor light. In a similar manner, variations in the detected signals due to componentry differences are largely eliminated through the use of a common detection configuration.

The optical switch 88 includes a signal input port 96 and “N” number of output ports 98, where “N” is an integer equal to or greater than two. The optical switch 88 is connected and adapted to receive light signals from the laser beam combiner 40 via an optical fiber 44, which fiber is connected to the signal input port 96. Each output port 98 is connectable to a sensor assembly 14 (e.g., via an optical fiber connector 46 as described above) for purposes of sending a light signal to the light signal outlet 17 of the respective sensor assembly 14. The optical switch 88 further includes a control signal port 100 for receiving control signals from a switch/multiplexer sequence control 101. These control signals determine through which output port 98 light emitted from the combiner 40 is directed.

The multiplexer 90 includes “N” number of signal input ports 102, a signal output port 104, and a control signal port 106. Each signal input port 102 is connected to a cable coupler 66 via electrical conduit 108. Each coupler 66 is adapted to connect to, and receive signals from, the shielded electrical conduit 64 of a respective one of the sensor channels 92, 94. The signal output port 104 is connected to signal processor 72. The control signal port 106 is adapted to receive control signals from the switch/multiplexer sequence control 101 that determine which one of the signals received by the input ports 102 is directed to the signal processor 72. As indicated above, in some embodiments a pre-amplifier 70 is disposed to receive the detector signals from the sensor assembly 14 prior to such signals reaching the signal processor 72. The signal processor 72 is adapted to process the detector signals from a given sensor assembly 14 (received via the multiplexer 90) in the manner described above, with the signals from each detector 20 processed independently of the signals from other sensor assemblies.

The signal processor 72 and the CPU or monitor processor 74 convert the received signals into physiological parameters by various spectrophotometric methods (e.g., those of U.S. Pat. Nos. 6,456,862 and 7,072,701), and the resultant physiological parameters (e.g., tissue oxygenation concentration or saturation levels) may be visually displayed on the user display 32. Also, light sampled by the laser output monitor photodiode 50 could be used as the input intensity (Io) signal utilized in spectrophotometric type algorithms such that described in U.S. Pat. No. 6,456,862.

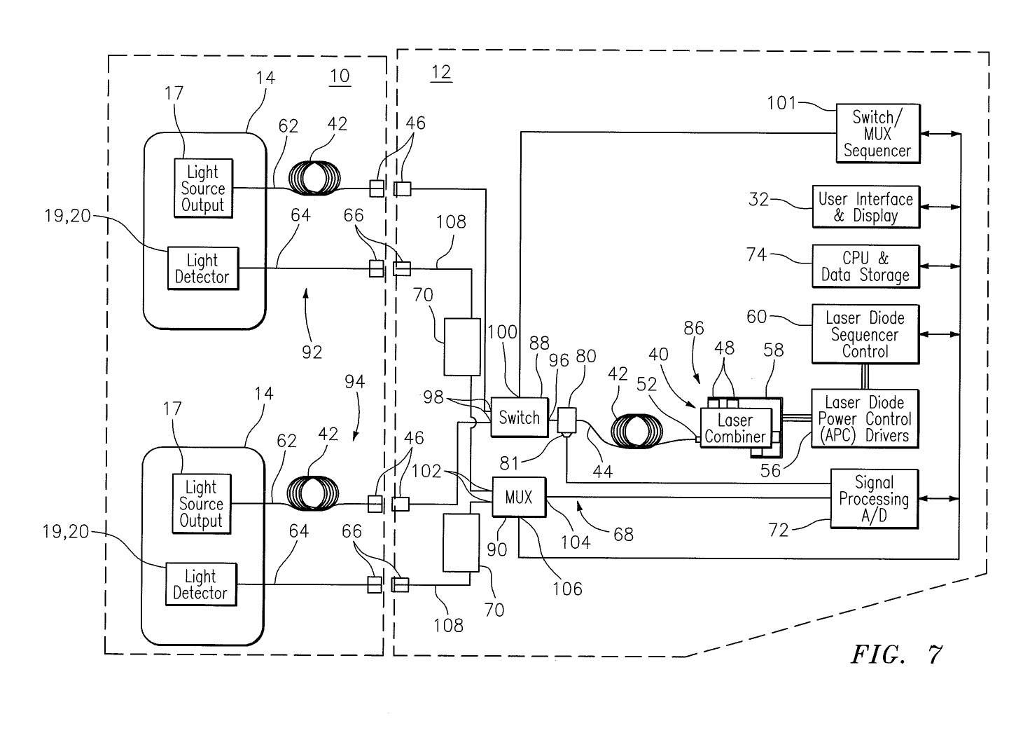

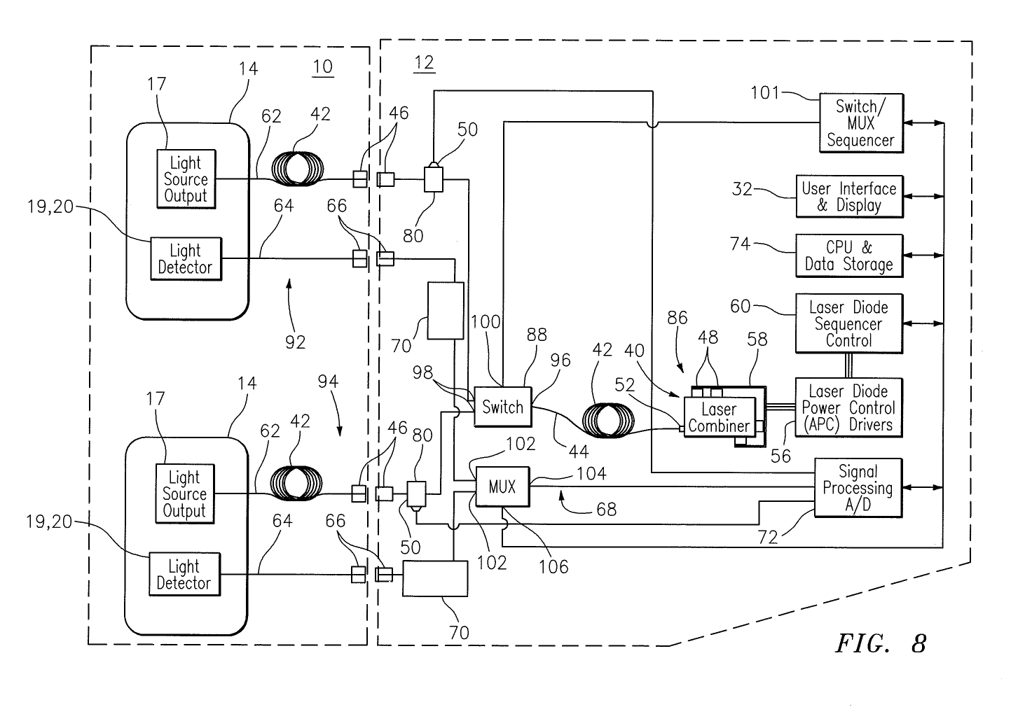

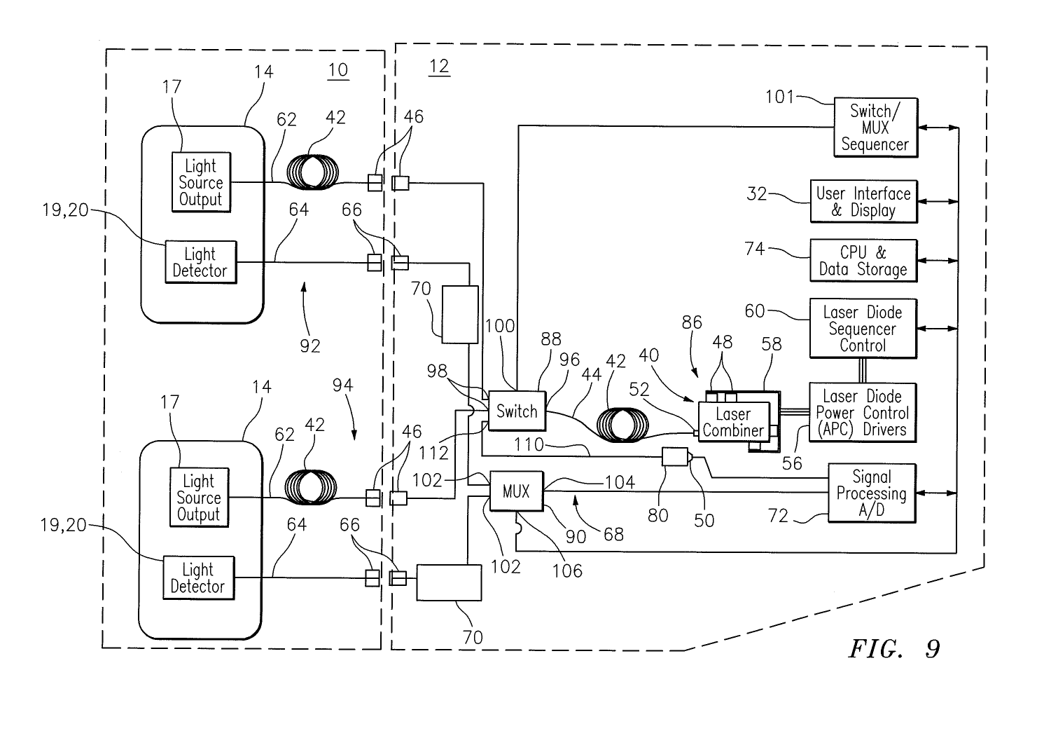

In the embodiment shown in FIG. 7, an inline laser output intensity monitor 80 is placed after the optical fiber light stabilizer 42 to sample light using a monitor photodiode 81, which functions in a manner similar to the laser output monitor photodiode 50 shown in FIG. 3. This arrangement is similar to that shown in FIG. 5. The inline laser output intensity monitor 80 contains a beam splitter that diverts a small percentage (e.g., ˜4%) of light to monitor photodiode 81. As indicated above, the diverted light signal can be used within the signal processor 72 (or CPU 74) as an input intensity (Io) signal, and the inline monitor optics can be used to increase the output NA of the light to be emitted from the sensor. In the alternative embodiment shown in FIG. 8, an inline laser output intensity monitor 80 is connected to or placed in-line with each output 98 of the optical switch 88. Each monitor 80 in this embodiment operates in the manner described above, providing a light signal back to the processor 72 and/or the CPU 74 and increasing the output NA of the light emitted from the sensor. In the alternative embodiment shown in FIG. 9, the optical switch 88 includes an extra channel 110, including an outlet port 112 (i.e., a “monitor outlet port”), dedicated to monitoring the light signal output to the sensor portion 10. An output intensity monitor 80 is disposed to receive light signals from the monitor outlet port 112. The monitor 80 in this embodiment operates in the manner described above, providing a light signal back to the processor 72 and/or the CPU 74.

Referring to FIGS. 6-9, in some embodiments, the sensor portion 10 may include an optical fiber light stabilizer 42 like that described above, disposed in line with the optical fiber cable 62.

Although the present invention has been illustrated and described with respect to several preferred embodiments thereof, various changes, omissions and additions to the form and detail thereof, may be made therein, without departing from the spirit and scope of the invention. For example, the present spectrophotometric system and method has been described above in detail in terms of a cerebral oximeter useful to determine the oxygenation of biological tissue. The present spectrophotometric system and method is not limited to the described cerebral oximeter embodiment, however, and can be used alternatively to determine other tissue characteristics, or used to determine the presence of other substances that can be spectrophotometrically identified.

What is claimed is:

1. A spectroscopic system that monitors oxygenation levels in biological tissue, comprising:
a sensor portion that includes at least one sensor assembly, which sensor assembly has at least one light signal outlet, and at least one light detector adapted to sense light and produce detected signals; and
a monitor portion having a processor in communication with the light detector in the sensor assembly, a light source adapted to produce light signals at a plurality of different wavelengths;
at least one optical fiber light stabilizer, wherein the light signals traveling from the light source, pass through the stabilizer where they are stabilized by redistributing modes, and subsequently pass through an optical fiber to the light signal outlet; and
wherein the processor is adapted to process the detected signals to determine oxygenation levels within the biological tissue.

2. The system of claim 1, wherein the optical fiber light stabilizer includes a multimode optical fiber wrapped around a spool.

3. The system of claim 3, wherein the optical fiber stabilizer has a length that is great enough to permit establishment of equilibrium modal distribution of the light within the multimode optical fiber.

4. The system of claim 1 wherein the optical fiber light stabilizer includes a fixture operable to introduce microbends within the fiber.

5. The system of claim 1 wherein the optical fiber light stabilizer includes a mode scrambler.

6. The system of claim 1, wherein the optical fiber light stabilizer is disposed within the monitor portion of the system.

7. The system of claim 1, wherein the at least one sensor assembly includes a first sensor assembly and a second sensor assembly, and the system further includes an optical switch with two or more output ports and an input port, wherein each switch output port is in light communication with the light signal outlet of a particular one of the first or second sensor assembly, and the switch input port is in light communication with the light source;
wherein the optical switch is adapted to selectively route light signals from the light source to each switch output port.

8. The system of claim 7, wherein the light source includes a plurality of laser diodes, and each laser diode is adapted to produce light signals at a predetermined wavelength, which wavelength is different from the wavelengths produced by the other laser diodes; and
wherein the system further includes a multiple laser beam combiner adapted to combine the light signals from the laser diodes into a single output light signal.

9. The system of claim 7, wherein the system further includes at least one output intensity monitor adapted to sample the light signals from the light source.

10. The system of claim 7, wherein the first sensor assembly and the second sensor assembly each include an optical fiber light stabilizer.

11. A spectroscopic system that monitors oxygenation levels in biological tissue, comprising:
a sensor portion that includes a plurality of sensor assemblies, each sensor assembly having at least one light signal outlet, and at least one light detector adapted to sense light and produce detected signals; and
a monitor portion having a processor in communication with each of the light detectors in each sensor assembly, a light source adapted to produce light signals at a plurality of different wavelengths, and an optical switch with two or more output ports and an input port, wherein each switch output port is adapted to be connected to the light signal outlet of a particular one of the sensor assemblies, and the switch input port is in communication with the light source;
wherein the optical switch is adapted to selectively route light signals from the light source to each switch output port; and
wherein the processor is adapted to process the detected signals to determine oxygenation levels within the biological tissue.

12. A method for spectrophotometrically determining an oxygenation level in biological tissue of a human subject, comprising the steps of:
selectively providing a plurality of laser light output signals each at a predetermined wavelength of light;
providing the laser light output signals to an optical fiber, where the laser light output signals propagate through the optical fiber;
stabilizing the laser light output signals within the optical fiber by redistributing modes of the laser light output signals until an equilibrium mode distribution is established in the laser light output signals propagating within the optical fiber;
emitting the laser light output signals into the biological tissue of the human subject;
sensing for the laser light output signals after they have passed through the biological tissue of the human subject, and producing detected signals corresponding to sensed laser light output signals; and
determining the oxygenation level in the region of biological tissue of the human subject using the detected signals and a processor adapted to process the detected signals to determine the oxygenation level.

13. The method of claim 12, wherein the step of providing the plurality of laser light output signals includes providing a plurality of laser diodes, wherein each laser diode selectively produces laser light output signals at a wavelength of light different from the wavelengths of the other laser diodes.

14. The method of claim 13, further comprising the step of combining the laser light output signals from each of the plurality of laser diodes into a combined laser light output signal, which combined signal is provided to the optical fiber.

15. The method of claim 13, wherein the step of stabilizing the laser light output signals includes passing the laser light output signals through a multimode optical fiber wrapped around a spool.

16. The method of claim 15, wherein the optical fiber stabilizer has a length that is great enough to permit establishment of equilibrium modal distribution of the light within the multimode optical fiber.

17. The method of claim 15, further providing at least one sensor assembly, which sensor assembly has at least one light signal outlet, and at least one light detector adapted to sense light and produce detected signals; and
wherein the laser light output signals are stabilized before the laser light output signals enter the sensor assembly.

18. The method of claim 12, further providing a first sensor assembly and a second sensor assembly, wherein each sensor assembly has at least one light signal outlet, and at least one light detector adapted to sense light and produce detected signals; and further providing the step of
switching the laser light output signals between the first sensor assembly and the second sensor assembly.

19. The method of claim 12, wherein the step of stabilizing the laser light output signals includes stabilizing the laser light output signals after the laser light output signals have been switched.

20. The method of claim 12, further comprising the step of monitoring the output intensity levels of the laser light output signals from the light source.
